(19) United States
(12) Patent Application Publication
(10) Pub. No.: US 2025/0179271 A1
(43) Pub. Date: Jun. 5, 2025

(54) Safe Landfill Material, Bio-Assimilation and Conversion Methodologies and Formulae

(71) Applicant: Robert DeMatteis, Grass Valley, CA (US)

(72) Inventor: Robert DeMatteis, Grass Valley, CA (US)

(21) Appl. No.: 18/840,773

(22) PCT Filed: Feb. 23, 2023
(86) PCT No.: PCT/US2023/013742

§ 371 Date: Aug. 22, 2024

Related U.S. Application Data
(60) Provisional application No. 63/314,026, filed on Feb. 25, 2022.

(51) Int. Cl. C08K 5/00; C08K 3/012; C12P 5/02

(52) U.S. Cl. CPC C08K 5/0033; C08K 3/012; C12P 5/02

(57) ABSTRACT

Disclosed herein are a number of methods, compositions, formulae, and closed loop systems for delaying the bio-assimilation of products constructed of at least one plastic-based material component and buried within a biological environment to prevent the premature release of biogases while also enhancing a subsequent biogas capturing period once the bio-assimilation period is triggered. The end result of bio-assimilation of the plastic material constructed in accordance with the principles of the present invention in a landfill is biogas that can be safely captured, a little water vapor, and biomass, which is returned to earth. It is a true carbon neutral closed loop synthetic plastics that began as natural gas may be turned back into renewable natural gas with little, perhaps no loss.

### CROSS REFERENCE TO RELATED APPLICATIONS

This application claims the benefit of U.S. Application No. 63/314,026, filed on Feb. 25, 2022, entitled Safe Landfill Material Degradation Methodologies and Formulae, and which is hereby incorporated by reference in its entirety.

### BACKGROUND

### 1. Field of the Disclosure

The present disclosure relates generally to materials, processes, and systems used to manufacture and sell various disposable and durable products, with a primary focus on non-recyclable plastics (referring to traditional hand-sorting and mechanical recycling systems), which plastics are primarily synthetic (some may include bio components), all of which may safely bio-assimilate and be converted into one or more biogases in a closed loop recovery system.

### 2. Background Art

All products whether they are disposable, short-term durable goods, or long-term durable goods, have a desired end of life (DEOL). In other words, all products eventually reach the end of their usable life—and, within the United States of America, most wind up in an EPA regulated landfill. Herein, the DEOL is considered the timeframe from the date of manufacture of the product to the product's final last use prior to disposal in an EPA regulated landfill, many of which have engineered biodigester environments that capture biogas from organic waste and sent to adjacent biogas plants for additional processing. Present methodologies and material formulae do not correctly address the DEOL and EPA regulated landfill standards for recovery. Even paper and cellulose-made products may fail in this regard when the products are disposed as waste. Compostable products, such as most bioplastics, are made to be composted and not discarded in the waste stream. And most accelerated degradation additives—either bio- or mineral-degradation-used in plastics do not have specific, safe degradation properties for EPA landfills. Further compounding the issues is that some of these accelerated-degradation products are incorrectly labeled as being recyclable, which is not in compliance with Federal, state (such as the state of California) laws, nor the standards and guidelines established by the Federal Trade Commission (FTC), the Sustainable Plastics Coalition (SPA), American Plastics Recyclers (APR), the Bio Plastics Institute (BPI) and others.

Degradation additive suppliers and their plastic film and bag manufacturing customers unfortunately apply a guesswork approach on the amount of additives used, usually as a percentage of the overall plastic feedstock(s). It is clear there is no one-size-fits-all additive, or formula, due to the complications associated with a product's DEOL, along with the essential requirements mandated by the U.S. Clean Air Act of 1970, to prevent the release of greenhouse gases (GhG), which includes but is not limited to methane, in EPA regulated landfills. One technology that uses a mineral salt, attempts to calculate usage on an end-of-life basis is revealed in U.S. Pat. No. 9,617,397 Tandio, et al. However, the ′397 Tandio patent only partially addresses the degradation issue. What is missing from the ′397 Tandio patent are specific applications, limitations and degradation time durations based on the EPA's landfill standards per the 1970 mandate. Likewise, the ′397 Tandio patent only partially addresses traditional manufacturer, distributor, and user methodologies, including storage, use, and their related time frames.

Oxo-degradables made by companies such as EPI and Symphony, have no specific DEOL considerations, other than perhaps approximate timeframes governed by their Cadmium based technology. This is an attempt to provide a one-size-fits-all approach. What is provided is a general time frame in which a plastic product may degrade-partially or completely-without accurate consideration for its DEOL or the EPA's landfill standards and complex environment. One reason for this, is their Cadmium additive degradant is known to work as a generally short-term oxidizing degradant (less than 3-4 years) and has uneven unreliable results in longer time frames. In other words, for a longer DEOL, the functionality of Cadmium salts is inconsistent producing partial degradation to some portions of the plastic, full degradation to other portions, and no degradation to yet other portions. The use of these Oxo salts and other bio- or mineral-salts, along with enzyme additives used to initiate degradation, are typically employed in a one-size-fits-all approach, none of which conform to the requirements of the large number of market segments using non-recyclable plastics, nor the requirements of EPA regulated landfills. It is also important to note, that it is during these rapid degradation time periods that the plastic deteriorates creating microplastic fragments and the releasing of GhG.

There is also a serious disconnect in commerce between the opinions and judgments of consumers, users, retailers, plastics manufacturers, additive suppliers, and environmentalists, and landfill operators in general. It is important to note that the U.S. EPA regulated landfills fall into two categories. First, regular landfills that are covered in an attempt to stop degradation and likewise trap the release of GhG per the 1970 Clean Air Act mandate, and; second. LMOP landfills, which prevent degradation for the most part and help trap the premature release of GhG under its coverings, then drill wells to initiate bio-assimilation of the organic matter, thus generating biogas that may then be converted into energy. While LMOP landfills represent about 30%-35% of the EPA landfills, they tend to be in larger population regions and process far more trash than non-LMOP landfills.

The disconnect in commerce also includes what should or should not be recycled in established recycling systems. For example, T-shirt bags returned to supermarkets are actively being recycled by large recyclers for other subsequent uses, which is not a true closed loop. Similarly, PET bottles that carry deposits have been actively recycled for many years. For products that are not recycled by conventional means today-referred to as “non-recyclables”—there are few standards. Many of the non-recyclable plastics used in commerce in the U.S. are contaminated with bacteria (common in food service and restaurants), are laminated to paper (such as freezer wrap), or are laminated with dissimilar materials (i.e., common pouch manufacturing for retail sales products).

Most single-use disposable products, such as trash bags, food containers and food service products, such as sandwich wraps, containers, and so on, that are contaminated with bacteria, are considered non-recyclable. Such single-use disposable products have a DEOL that would perhaps be 6 months to 2 years, including the time period before they are even used by a consumer, then used by the consumer, and afterward disposed in the trash. While most single-use disposables are used in a relatively short time frame, others may be stored in distributor warehouses for longer periods of time, or in a user's inventory, thus a 2-year DEOL time frame may be more appropriate. The DEOL also applies to packaging such as disposable plastic pet food bags, potato chip bags and meat wraps, with a DEOL most likely less than 2 years. After all, the food products contained therein would most likely spoil in a longer time frame anyway. Thus, most single-use film products are either contaminated with bacteria (food wraps), have chemical contamination (such as cement bags, construction films, etc.), and contain dirt and oils when thrown out into a well-used trash bin. They are considered non-recyclable. Yet, most single-use plastic T-shirt bags are considered recyclable and may be efficiently recycled, which is actually a form of “downcycling” the plastic into products that have inferior strength and sanitation qualities.

Short-term durable goods generally have a DEOL from 2 years up to perhaps 8-10 years. For example, inexpensive reusable food containers used in homes and some multi-layer retail pouches may have a 2- to 4-year DEOL. Inexpensive plastic utility buckets used around the home for painting or gardening, may have a 5-7 year lifespan. Plastic utility tarps may last up to 5 years outdoors and are often highly contaminated (oils, wood sap, dirt, dead bugs, and so on) and deteriorated from sun or light exposure. The DEOL of these durable goods may vary from 2-10 years depending on thickness, retardants (such as UV prevention additives), how often, and where they are used (indoors or outdoors for example). Multi-layer pouches and durable films used for packaging and wrapping various foods and durable goods (paints, motor oil, and so on) may best have a DEOL of at least 5-8 years. Plastic products in this category do not meet present day recycling requirements.

Long-term durables may have a DEOL from 10 years to 20 years long. This may include lawn chairs with a DEOL of perhaps 10-15 years, or plastics used with certain garden ornaments with a DEOL of 15-20 years. Even though high quality Tupperware® is intended to be reused for several years, they eventually lose their functionality (memory), and can no longer be sealed airtight after 5-10 years or so.

Long term durable synthetics over 25 years or so, may include plastic fencing, pipes, and synthetic wood-replacement goods, like lumber, pallets, and benches. For such products, it would be desirable to have no DEOL. These products may be recycled after extensive use with their physical properties partially restored in the re-pelletizing process with new polymers, and afterward reused once again for new products. Note that many of these long-terni durables are made with recycled plastics, such as grocery sacks. Another group of products that have long DEOLs in excess of 25 years include heritage-type films and containers used to envelop and preserve antiquities, such as coins, antique rugs, and museum artifacts. These heritage-type items are usually stored indoors and resist UV light or exposure to the elements.

Based on their normal degradation qualities in a biodigester such as a landfill, most standard plastics such as polyethylene, PET, BOPP, and other synthetics, may have an estimated 100 to 150-year life. In the case of compostable plastics, they break down into smaller pieces in perhaps only 90-180 days in industrial composting facilities. When degradation occurs, neither synthetic nor compostable plastics meet EPA LMOP landfill requirements, and both produce unwanted greenhouse gas (methane. CO2, and others). It is obvious that the 250-year life of a plastic item buried in a landfill, literally allows the greenhouse gas to trickle out over a very long period of time. In sharp contrast, the compostable plastics immediately release greenhouse gas into the environment and may affect the premature degradation and release of GhG in the surrounding trash in an EPA LMOP landfill. From an environmental perspective the emissions of GhG are the primary cause of climate change and, both extremes are problematic contributing factors.

The Environmental Protection Agency (EPA) reports that the third largest manufactured source of greenhouse gas production in the U.S. comes from its landfills. This is caused by organic trash that degrades and releases GhG principally in the form of methane and CO2, which are commonly referred to as Landfill Gas (LFG). To prevent premature release of the gases, EPA landfills are covered in layers as the trash builds up. However, biodegradable products that are discarded and find their way to the upper tier of an open landfill, may initiate degradation forthwith, prematurely releasing GhG.

In an attempt to reduce the greenhouse gas production in landfills, the EPA started the LMOP program in 1994. In summary, the Federal EPA sponsors landfill operators to safely capture the escaping LFG greenhouse gases and convert them into electricity, fuel, or some other form of energy. It is conceivable they could even use machinery to convert said gases right back into virgin plastics as anticipated in the future. The GhG extraction process is carried out by using biogas extractors such as drilling LFG wells, a series of tubes implanted beneath the landfilled trash initiating aerobic bio-assimilation of the organic matter. After about two years, the degrading organic contents begin anaerobic bio-assimilation and release LFG, whereas methane gas is generated that is captured and pumped through pipelines to an electric generation facility or may be stored for use as fuel or cleaned and converted into Renewable Natural Gas (RNG). These wells tend to be productive for about 30 years, thus ordinary synthetic plastics do not contribute to the capture and use of the LFG. The materials that promote degradation are principally paper, wood, cotton fabrics, food stuff and so on, which degrades during the 30 year usable life of an LMOP landfill.

It is interesting to note that at the initiation of the LMOP program, greenhouse gas emissions from landfills were the second largest manufactured source. From 1994 to 2013, about 30%+ of the 2122 EPA landfills have been converted into LMOP landfills with the positive impact of fugitive landfill gas being reduced and thus, becoming the third largest manufactured source. With the ultimate conversion of all EPA landfills capturing LFG to convert it into some form of energy (or otherwise), LFG emissions from landfills may become an insignificant source of greenhouse gas escaping into the atmosphere in the years to come, a major step to preventing global warming. LFG well drilling is becoming the preferred means of landfill treatment systems as this process not only safely captures GhG and generates profit, but such process helps a landfill to properly settle, hopefully avoiding slippage that can cause landslides. Furthermore, some countries that have been promoting the use of GhG emitting Oxo-degradables, are now gearing up their future to convert their landfills to the United States LMOP model and convert the extracted GhG into usable energy or RNG.

In US landfills and many others worldwide, landfill trash is compacted by heavy equipment bulldozers. Obviously, this is to minimize the space requirements and likewise assist in avoiding slippage of its contents over time. Water, erosion, and other land movement factors can contribute to landslides, which may have devastating results if the landfill is near a populated area or a river. The compacted trash is periodically covered with plastic sheeting, which significantly prevents air and moisture from reaching trash and helps prevent the upper layers of landfill trash from premature degradation, and the undesirable release of GhG. Landfill operators want to prevent all degradation in compliance with the EPA mandate under the Clean Air Act of 1970 and allow the contents to safely degrade only after LFG wells are drilled. However, one significant problem that the practice of covering has created, is that the trash in the upper layer that has begun premature degradation is also covered. More trash is then filled atop the first cover, compacted, and then covered once again, and so on with multiple layers (for several years), all of which may have some trash that is prematurely degrading. In doing so, and with the aid of aerobic and anaerobic digestion from certain plastic products (i.e., rapid degradation from ASTM D5511 plastic products and perhaps oxo- and enzymatic degradables) is forcing the premature release of GhG out sideways. In other words, the pressure from under-cover LFG gas created by the premature degradation of trash, forces the GhG to escape sideways contaminating adjacent lands, and then upwards into the atmosphere.

In an LMOP landfill there are three potential (and primary) means of the premature release of GhG that avoids safe capture. Firstly, the trash (but not most plastics) near the upper surface can initiate degradation in each compacted and covered layer before the landfill is filled anew with trash and covered once again. It is essential to note that covering and layering trash in LMOP landfills goes on for years. Typically, LFG wells may not be drilled until after four years of depositing trash in the first, covered lower layer. The surface trash that has initiated degradation consists of a majority of organic matter and compostable materials (including bioplastics and the like), paper products, food stuff and food residues, and moisture, etc., and prematurely releases GhG. Secondly, should an anaerobic agent be blended with a plastic material deposited in the trash stream, such agent can also initiate premature degradation, thereby releasing greenhouse gases. An example of said anaerobic additive is Neo Plastics (Aripack, Inc.), which reportedly begins to anaerobically degrade upon arrival to a landfill, which contributes to premature release of GhG. Premature degradation also contributes to a lower output of the desired methane production later on in the LMOP landfill biogas production process. Thirdly, the erratic oxo-degradable additives blended with a synthetic plastic can also contribute to the premature degradation and the premature release of greenhouse gas. These degradation means, along with other lesser used degradation accelerants, contribute to the sideways escape or GhG through adjacent lands. Once degradation begins, degradation migration to other materials that would not have otherwise initiated premature degradation in the covered environment may also occur. These three common landfill conditions contributing to the premature release of GhG pursue the folly of a supposed requirement that all materials must rapidly become biodegradable.

Another outcome of the degradation of various organic and synthetic materials and plastics in landfills is the potential release of toxins. For example, compostable plastics are made from predominantly genetically modified (GM) substances and include genetically modified organisms (GMOs). These are touted by some health organizations as contributing factors to cancer. Compostable plastics also contain “genetic herbicides” that largely has an unknown effect, other than reports that they cause watershed pollution, soil contamination, and contribute to the production of super weeds. GM corn stocks may also be contaminated with commercial pesticides or are irrigated with contaminated water. Oxo-degradable additives and anaerobic additives likewise may contain heavy metals, such as cadmium that are considered highly toxic in higher concentrations. Last, printing inks have some degree of toxicity, even when water based. Generally speaking, paper products use more ink than plastic products, contributing more toxicity to the degrading content, and ultimately into the soil and possibly the air along with the fugitive GhG. Regardless of the source of potential toxins released in landfills, it is desired to minimize their effect.

One example of a company that sought to reduce these problems is ECM Biofilms. Plastic products made with its ECM MasterBatch Pellets will reportedly biodegrade in a “biologically active” environment (such as may occur in a landfill) in a time frame initiated after one year. Such products may degrade due to aerobic and anaerobic digestion, and degradation may be encouraged when in close contact with other degrading contents. Unfortunately, this means there is no control of the additive in a landfill, since at least some early biodegradation occurs, especially in the upper strata. Thus, this approach also promotes premature degradation and premature release of GhG. This further promotes the early degradation of nearby contents that would otherwise stay dormant until LFG wells are drilled. With no consideration of DEOL based on product usage, storage, etc., and with no consideration of the four year time period before drilling LFG wells in EPA LMOP regulated landfills, other than perhaps in the final, last layer, this approach promotes the premature degradation and the release of fugitive GhG, along with minimal, if any, contribution to the subsequent extraction of desirable biogas.

Various plastic materials deposited in U.S. landfills have a hodgepodge of degradation properties, life spans, contamination issues and toxicity. There is little consistency of standards employed by the plastics trade for non-recyclable plastics, other than those listed by APR, which few manufacturers are able to apply. Worse yet, the degradation properties of plastic products sold and used in the multitude of over one hundred major market segments is not considered. There may be considerations for the durability and performance of plastic products in the various market segments, but there is no consideration for its safe degradation of products disposed in landfills, principally US EPA regulated LMOP landfills.

Since the inception of the Clean Air Act in the United States in 1970, the initiation of LMOP in 1994, it is apparent that many good intentioned entities have pursued—in many cases mistakenly-degradation issues that have actually increased the release of GhG instead of preventing it and have increased the release of toxins instead of reducing them. The overabundance of technologies at best, only partially address the GhG and toxin issues. The many additives, various types of plastics, and performance requirements in the many market segments, only complicates the problem of safely capturing greenhouse gas in landfills as mandated by the EPA. It is understandable that after 50 years of the passing of the Clean Air Act, and after 25 years of building LMOP recovery systems, the current methods are inadequate, and at times are dangerous, self-defeating even, which includes compostables and bioplastics. Adding to the impact of the misguided methodologies is the lack of public knowledge of knowing what to do and what to buy. Consumers at best are exposed to incomplete scenarios as to the true effects on the environment of various types of degradable technologies, let alone what actually occurs in a landfill. It is a fact that degradable plastic suppliers and manufacturers avoid LFG applications and premature greenhouse gas issues altogether. For the most part, they are unaware of what happens in a landfill, let alone an EPA or EPA LMOP landfill. These issues, problems, are of great concern to EPA landfill operators, federal and state governments, and humanity in the long term.

Right at the heart of the problems created in EPA LMOP landfills is clearly the obsession for the plastics trade and environmentalist to incur rapid degradation of plastics, which is clearly not desirable in landfills. Another huge problem is due to the sophisticated LMOP landfill engineering not being considered by the plastics trade. It is rare that plastic scientists have a background in landfill environments—LMOP or otherwise. If they do, such background tends to be incomplete. They do not consider the first four years of compaction and covering and two subsequent years of aerobic digestion. The obsession to reduce a plastic “back to dirt” is a wrongful concept. Most plastics in the U.S. come from a natural gas feedstock—unlike bioplastics (grown in dirt) and why should it be turned into “dirt” instead of a valuable biogas to produce energy, or better yet, RNG. To have a true closed loop on synthetic plastics, the bio-assimilation should result in the conversion back into natural gas. In other words, Renewable Natural Gas (RNG) is a desired end product of the bio-assimilation process. All other recycling attempts are nothing more than downcycling at best, into other lesser quality plastic products.

A plastic with a DEOL scenario that overcomes the numerous problems associated with prior art and would subsequently, controllably degrade in a biodigester environment such as an LMOP landfill in a timely manner to prevent premature release of GhG, and subsequently be captured by LFG type wells and profitably converted into biogas and RNG, would be valuable worldwide—to landfill operations, manufacturers, users, and consumers. Materials specifically formulated with standardized properties, using essential bio-assimilation guidelines that meet the EPA's LMOP landfill requirements as trash disposed therein is converted to energy, prevents the premature release of GhG, and leaves minimal toxic residues is highly desirable. Furthermore, plastics using this approach would be consistent with present day recycling goals, since the products ending out in landfills are considered contaminated or non-recyclable in one way or another. When safely bio-assimilated during the life of an LMOP landfill, the end result is a compact, stable, landfill that contains little or no residue minerals or toxins left in the soil. Furthermore, the proposition to incorporate such a plastic that meets manufacturer, user, and consumer requirements, and which may be verified by suitable ASTM tests, and an NGO or government chain of custody to verify its claims of non-toxicity, and meeting EPA LMOP requirements, can result in a true closed-loop recovery system.

### SUMMARY

Embodiments constructed in accordance with the present invention overcome one or more of the problems associated with the prior art and effectively resolve long standing, rather serious, problems in landfills with disposable plastics. Furthermore, embodiments constructed in accordance with the principles of the present invention does this cost-effectively, uses the existing infrastructure, and have obvious health and environmental benefits. Little to no landfill GhG, including CO2 and methane gases, are generated until after LFG wells are drilled, whereas degradation of the plastic material may begin—and the resultant biogas may be safely captured and converted to energy or RNG. The end result of bio-assimilation of the plastic material constructed in accordance with the principles of the present invention in a landfill is biogas that can be safely captured, a little water vapor, and biomass, which is returned to earth. It is a true carbon neutral closed loop—synthetic plastics that began as natural gas may be turned back into renewable natural gas with little, perhaps no loss.

The best environment to utilize the embodiments constructed in accordance with the principles of the present invention is in EPA LMOP landfills, which are ideally suited to a closed-loop system when used in conjunction with the embodiments described herein, in landfills that do not have LFG to energy systems, the embodiments constructed in accordance with the principles of the present invention and remain dormant with a time bomb effect, until such time as the landfill may be converted to process the LFGs, such as in EPA LMOP landfills in the United States.

The embodiments constructed in accordance with the present invention are primarily for use with disposable plastic products that are not candidates for conventional recycling systems. Such embodiments are also ideal for plastics that have inadequate recycling infrastructure in a given region, for example, in much of California. In other words, the embodiments constructed in accordance with the present invention are for non-recyclable plastic products, which will find their way to a landfill including single use disposables, along with short-term and long-term durables. The embodiments disclosed herein also aim to connect the dots for those products to have sound environmental qualities and meet the U.S. EPA LMOP landfill standards. The solutions described herein combine a technology that creates unique solutions using emerging technologies that create an interrelated thread through humanity, society, and commerce—with sound, clear-cut long-term environmental outcomes, preferably in a true closed loop. The solutions disclosed herein also draw upon the industrial arts, engineering, and applied sciences to create practical solutions that meet existing and anticipated federal, state, and municipal laws, and are in full compliance with the objectives of the EPA, FTC, APR, SPC, and so on.

In doing so, the landfill-safe technologies revealed herein provide methodologies that determine a suitable end of life (DEOL) for a material, most importantly plastics, which likewise concur with preferred EPA LMOP landfill bio-assimilation requirements. The embodiments disclosed herein also further provide for systems and processes that use non-toxic additives, molecular modification, and blending methodologies tailored to a specific product's defined usage, and which likewise conforms to the desired standards set forth in EPA landfills. The systems and processes disclosed herein may also include the use of additives and molecular modification methodologies and systems integrated into the resin manufacturing process.

One or more embodiments disclosed herein: 1) employ the use of present day standards to properly designate a product's approximate DEOL; 2) are generally based on established time-driven standards of usability (single-use, short-term and long-term durables), and; 3) have a predetermined objective to not allow or initiate any form of degradation for a period of at least 4-5 years afterward expiration of the DEOL, thereby preventing the premature release of GhG, and; 4) only after present invention products have been deposited and covered in a landfill and the LFG wells have been drilled, shall bio-assimilation commence under influence of the common microbes found in EPA landfills, and; 5) with essentially full bio-assimilation taking place at or prior to the expiration of the landfill life of the covered and LFG well-drilled landfill, preferably 25-30 years max, leaving little to no trace of plastic-based material while also producing collectible biogas in a final bio-assimilation environment. By doing so, plastic products constructed in accordance with the principles of the present invention avoid generating GhG until after bio-assimilation begins in the biodigester environment and contributes to the generation of biogas used to generate energy in a nearby electric generation (or fuel conversion) or RNG conversion facility. In addition, the biomass and water that is created during the plastic bio-assimilation process becomes a non-toxic, non-liability component of the soil.

The preferred outcomes using the systems, processes, and materials as described by one or more embodiments described herein in accordance with the principles of the present invention may include the use of one or more non-toxic bio- or mineral-salt, or organic or inorganic substances, as may preferably be referenced. In addition, or as an alternative thereto, such systems, processes, and materials may include the use of molecular modification and embedded additives during the resin cracking process. The additives incorporated in the plastic material shall likewise meet RoHS. TRI and DTSC toxicity standards. It is also preferable to include product markings that comply with state and federal laws for consumers as, for example, a Do Not recycle notice required by the state of California. It is also desirable for the plastic manufacturers and suppliers to have test procedures conducted under specific ASTM guidelines. It is further desirable to have a verification system conducted by a state agency or an NGO for on-going compliance. Furthermore, it is desirable to establish a new ASTM standard that incorporates the use of existing test methods to verify the various stages on the overall non-degradation and subsequent bio-assimilation timeline that meet the EPA LMOP requirements, and optionally an agricultural organic biogas plant's requirements.

Key aspects of the embodiments constructed in accordance with the principles of the present invention-unlike prior art-allow mother nature in an LMOP landfill to do its job. The obsession of rapid degradation of prior art—is obviated by the ability of the embodiments disclosed herein and constructed in accordance with the present invention to slow down, even postpone, bio-assimilation of a plastic material so it may safely be converted into biogas—and energy or RNG. With non-toxic bio- and mineral-salt (organic and inorganics substances) additives, it is a matter of using generally evenly distributed, embedded quantities, preferably with generally smaller particle sizes, which formulae match the engineered conditions of an EPA LMOP landfill. It is also important to note that formulae concepts introduced for use in EPA LMOP landfills, may be adjusted to other plastic products, for example agricultural films, to meet the engineered requirements in biodigesters used in farms, dairy operations, even waste food digesters.

Overall, the inability of the existing fossil fuel plastics trade to “connect the dots” is surprising—and clearly verified by its obsession that rapid degradation is somehow beneficial to the planet, as well as its pursuit of recycling technologies that are nothing more than dirty, bacteria-ridden, downcycling operations. Alternative methodologies, for example, pyrolysis and chemical recycling, have been banned in the state of California due to toxicity issues. In the trade it is considered a costly method to convert “old plastic” into a costly gas such as propane. Why? In sharp contrast, this disclosure explains systems, processes, and materials for naturally and inexpensively converting plastic right back into its original, versatile feedstock natural gas, which can be forever renewed as RNG. All of this is accomplished using the existing waste management infrastructure with no health or safety issues whatsoever.

The embodiments disclosed herein may support one or more of the following objectives but are not limited to:

1. A system to modify a plastic product's properties to safely bio-assimilate in EPA LMOP landfills.

2. A plastic degradation system that does not prematurely release greenhouse gas when disposed in landfills.

3. A plastic degradation system that initiates bio-assimilation in LFG landfills at least four years after being deposited, compacted, and covered in LFG well landfills.

4. A system that fully bio-assimilates plastic prior to the 30 year LFG landfill life.

5. A system and process to manufacture plastics and plastic products to bio-assimilate in LFG wells beginning at a future timeframe (for example four years) after being deposited, whereas it is fully bio-assimilated by the end of the landfill's life (for example, 30 years later).

6. A system to manufacture degradable plastic products that would otherwise be considered contaminants in conventional recycling streams.

7. A system to determine an approximate DEOL based on traditional factors for example one of the following: A) Single-use disposable products; B) short-term durable products, and: C) long-term durable products.

8. A system to determine a DEOL based on environmental factors, for example in cold and very hot regions.

9. Essential formulae to manufacture plastics using bio- and/or mineral additive substances in plastics to create desirable bio-assimilation properties.

10. Landfill-safe formulae that meet the standards and requirements of the EPA, FDA, FTC. APR, SPA, and all other relevant laws and agencies in a given region, state, or nation.

11. Landfill-safe formulae and related processes used to create a plastic material as cited herein.

12. A process to manufacture plastics by blending plastic materials that results in bio-assimilation properties that meet landfill objectives.

13. A process to manufacture plastics by molecular modification resulting in desired bio-assimilation properties as cited herein.

14. A process to manufacture plastics using chain extenders, to extend the desired life of a product falling under the present invention to be safely disposed in a landfill.

15. A process to manufacture plastic products using bio- and mineral-additives in blended portions that are considered non-toxic when used in a plastic product, and subsequently initiates fragmentation and subsequent bio-assimilation after an LFG well is drilled.

16. A process to manufacture plastics using one or more of the following minerals as an additive or blended portion, to meet the bio-assimilation requirements of the present invention, but not limited to: A) cobalt; B) iron; C) cadmium; D) calcium; E) magnesium; F) calcium oxide; G) calcium carbonate, and; H) stearate variations thereof.

17. A process to manufacture plastics and plastic products using a starch or any number of organic substances (grown crops, extracts, waste, and so on) as an additive or blended portion to initiate, contribute to, or provide food to aid bio-assimilation to meet the desired time frames, for example, but not limited to: A) tapioca; B) avocado (and/or their seeds); C) beets; D) sorghum; E) potatoes; F) agave, and; G) corn.

18. A process to manufacture a plastic using one or more of the following biopolymers as an additive or blended portion to meet the bio-assimilation requirements of the present invention, for example, but not limited to: A) chitin; B) chitosan, or; C) commercially manufactured biopolymers for example, Ecoplas™ and Ingeo™ plastics.

19. A process to manufacture a plastic by a combination of one or more of the following: blending materials, introducing additives, and physically modifying the molecular structure so that it has time-sensitive bio-assimilation properties as cited herein.

20. A plastic that is formulated to be converted into biogas.

21. A plastic that is formulated to resist biodegradation and promotes conversion into biogas by way of bio-assimilation in a biodigester environment.

22. A plastic product made according to the processes and methods cited herein that inhibits early degradation and the release of one or more GhG, such as carbon dioxide and methane, until after the LFG well is drilled and bio-assimilation begins.

23. A plastic product that degrades according to the processes and methods cited herein that inhibits the release of one or more GhG, such as carbon dioxide and methane, up to the first 4 years that helps prevent the premature degradation of adjacent landfill contents.

24. A plastic product that degrades according to the processes and methods cited herein that helps improve bio-assimilation of landfill contents in the waning years prior to the end of an LMOP's well's life, thereby improving biogas production profitability.

25. A plastic product that degrades according to the processes and methods cited herein to begin bio-assimilation at least 4 years after being deposited, and that is fully converted into biogas no more than 30 years afterward.

26. A plastic product that degrades according to the processes and methods cited herein and complies with EPA LMOP standards, as may be altered in the future.

27. A methodology to verify degradation and bio-assimilation (or biogas conversion) of a material so that it complies with the lawful standards of a given government or public agency, for example record keeping.

28. A methodology to verify that a material safely degrades in accordance with a landfill's standards so that the resultant LFG may be safely captured, for example ASTM tests.

29. A methodology to verify that a material safely degrades in accordance with a landfill's standards and the resultant LFG may be safely captured and converted into biogas in an LFG to energy landfill.

30. A methodology to verify the degradation of a material leaves no or a negligible toxic substances which are acceptable on the TRI and DTSC lists, for example, RoHs tests.

31. A methodology to verify that the degradation of a material as cited herein is based on generally accepted ASTM guidelines.

32. A compliance system that verifies compliance with manufacturers of feedstock, additives, and plastics used to make products falling under the landfill-safe technology of the present invention.

33. Any one or more of the items on this list cited herein using a select group of catalyzer additives, but not limited to such.

34. A unique catalyzer formulated by pyrolysis, including a calcium oxide, or others.

Furthermore, it is an object of this application to illustrate the preferred methodologies, processes, formulae, and plastic compositions and broadly state the various ways the present invention may be efficaciously applied in the various trades.

All of the embodiments summarized above are intended to be within the scope of the invention(s) herein disclosed. However, despite the discussion of certain embodiments herein, only the appended claims (and not the present summary) are intended to define the invention. The summarized embodiments, and other embodiments and aspects of the present invention, will become readily apparent to those skilled in the art from the following detailed description of the preferred embodiments having reference to the attached figures, the invention not being limited to any particular embodiment(s) disclosed.

### BRIEF DESCRIPTION OF THE DRAWINGS

The accompanying drawings, which are incorporated herein form a part of the specification, illustrate the concepts of the present invention and, together with the description, further serve to explain the principles of the invention and to enable a person skilled in the pertinent art to make and use the invention.

FIG. 1 is a chart illustrating an approximate long-term timeline and lifespan of a single-use plastic product constructed in accordance with the principles of the present invention after disposal in an LMOP landfill.

FIG. 2 is a chart illustrating an approximate short-term timeline and lifespan of single-use plastic product constructed in accordance with the principles of the present invention after disposal in an LMOP landfill.

FIG. 3 is an exemplary block diagram of a set of closed loop recycling components of the safe landfill, delayed bio-assimilation system constructed in accordance with the principles of the present invention.

FIG. 4 is a flow chart illustrating how an exemplary plastic constructed in accordance with the principles of the present invention is discarded at the end of its usable life and converted to biogas and energy or RNG in a landfill.

FIG. 5 is a block diagram illustrating the requirements that may be applied to correctly produce products made in accordance with the principle of the present invention.

### DETAILED DESCRIPTION

### A. Description of Exemplary Products, Methods of Use, and Processes

In general terms, the present disclosure relates to systems and methodologies for producing products incorporating a plastic-based material component that may be safely bio-assimilated within a biodigester such as an Environmental Protection Agency (EPA) regulated Landfill Methane Outreach Program (LMOP) landfill or other related disposal systems, such as organic biodigester plants, such as those common in the agriculture trade. As opposed to rapid degradation focused technologies obsessed with turning products into dirt instead of RNG, the bio-assimilation timing disclosed herein is preferably delayed to take place in a timelier manner in order to prevent premature release of greenhouse gases (GhG) that may then be captured after the delayed production and converted into one or more biogases and further into an energy source or RNG. The present disclosure also provides for a public awareness marking system allowing consumers and users to be informed of product life expectations prior to disposal. Furthermore, the present disclosure provides for methodologies to test and verify claimed bio-assimilation properties of the material to meet desired EPA landfill properties and likewise ensure compliance with federal and local laws.

The examples used in the detailed descriptions are generally for non-recyclable (referring to the well-known mechanical sorting and recycling systems) plastic products, regardless of their 30 being of single use or have a longer DEOL. The following word combinations should be interpreted interchangeably: A) additive and compound; B) salt and substance; C) bio-and organic; D) mineral and inorganic, and, E) biogas, LFG, and GhG. In addition, the term biodigester, as used herein, refers to LMOP landfills and other environments in which materials are disposed that undergo bio-assimilation under certain conditions. As used herein, bio-assimilation refers to the consumption by common microbes found in a biodigester environment during its lifespan. It is important to distinguish the difference with use of the word “biodegradable”, which word has been frequently misused and misinterpreted. To note, FTC Part 260 cites the use of the word with packaging refers to “degradation in a relatively short time period”, which the present invention does not want. During the dormancy period there would be little to no resultant microplastic. This is also true following completion of the bio-assimilation period in the biodigester. As part of the bio-assimilation process, GhG may be produced as a result of either aerobic or anaerobic processes, although the bulk of the greenhouse gas methane is produced as a result of an anaerobic process.

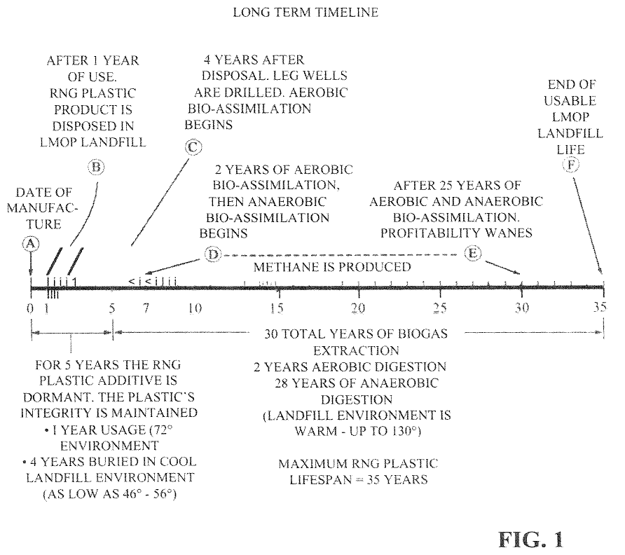

Referring now to a first exemplary biogas production and extraction scenario shown in FIG. 1, a long-term timeline and lifespan of a single-use disposable plastic product constructed in accordance with the principles of the present invention and the product's subsequent lifespan in an LMOP landfill is illustrated. As shown in FIG. 1, the product's useful lifespan begins at the event A, the date of manufacture. Following manufacture, the product's journey includes an approximate first year time period ending at event B during which the product undergoes shipping and storage in primary warehouses as well as subsequent storage at other locations such as packaging distributors, restaurants, supermarkets, and home pantries for example. Such time period also includes the end use by the consumer. It will be appreciated that such activities during this period typically take place at a “room temperature” of seventy-two degrees, although this is not meant to be limiting in any manner. These activities take place in an ambient environment external to the landfill/biodigester environment. Much of this single-use plastic is contaminated with foodstuff and dirt (for example, sandwich wrap, produce bags, poly-lined coffee cup, etc.). This one-year period ending at event B defines the products desired end of useful life (DEOL). After the product's DEOL expires, the product is discarded into an existing waste management trash pick-up stream and disposed at an EPA regulated LMOP landfill. Once delivered to the landfill, the product is typically compacted and periodically covered (buried) in cells (a specific location in the landfill) for a period up to four more years after the expiration of the DEOL and ending at event C. The compacting and covering (burial) process in the EPA LMOP landfill is carefully engineered to prevent as much GhG as possible from escaping into the environment (atmosphere). In this example a plastic article constructed in accordance with the principles of the present invention may be deposited in a new cell and remains dormant at the bottom as other trash, primarily organic content, is piled on top for up to four years as per LPA LMOP standards. Most trash in the U.S. is around 73% organic, and 27% is plastic. During this four-year burial dormancy stage or time period, temperatures are generally cool, as low as 45 degrees up to 55-60 degrees, some warm locations may be somewhat higher, and cool northern climes may be lower. After a four-year period ending at event C as measured from the DEOL event B, LFG wells are drilled placing one or more biogas extractors in communication with the environment defined by the landfill (biodigester) and connected to a pipe network. The decomposing trash that has built up a certain amount of biogas is released and flows into the pipe network (not shown). It will be appreciated that the burial dormancy time period between event B and event C is typically 4-5 years in an LMOP landfill, although this is not meant to be limiting.

For the next two years after the end of the five-year period beginning at event C, the buried trash begins to bio-assimilate more rapidly in a mostly aerobic and partially anerobic process. Around the end of seven-year period (event D) the bio-assimilation process transitions predominantly to an anaerobic process, which produces the desirable methane biogas which may be used to generate energy (electricity) or convert into RNG. Between the end of the seven-year period event D and the end of the 30-year period event E (i.e., a total of 23 years), the landfill profitably converts the methane as desired due to the triggering of the bio-assimilation process following the initial delayed or deferment period. From the end of the 30-year period event E to the end-of-usable-landfill life event F, the production of biogas wanes until production ultimately expires and collection is terminated. It is interesting to note that the embodiments disclosed herein in accordance with the present invention can indeed help extend this profitability timeframe. Likewise, it boosts output and profitability in any biodigester environment by convert the plastic of the present invention into biogas, that was previously not feasible.

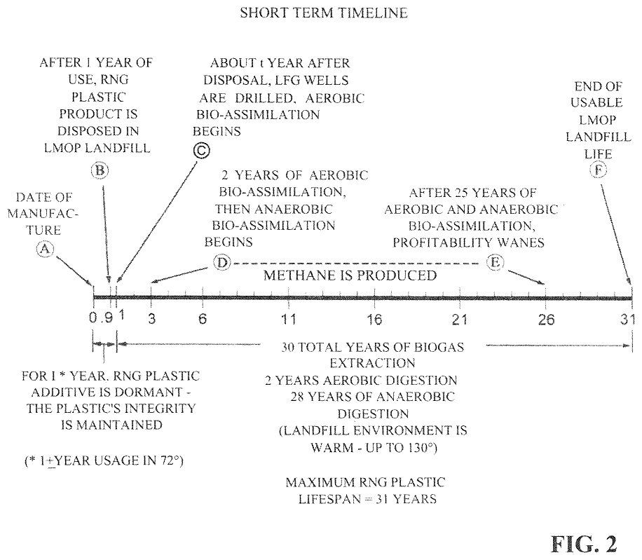

In the second exemplary biogas production and extraction scenario illustrated in FIG. 2, a short-term timeline and lifespan in an LMOP landfill for a product constructed in accordance with the principles of the present invention is similar to that of FIG. 1 with the same start of useful life at event A (manufacturing date). However, FIG. 2 illustrates a very short period of time that the product constructed in accordance with the principles of the present invention is buried and covered in a landfill as represented by the time period between first-year period ending at event B, when the product is first disposed in the landfill, and the one-year-plus period ending at event C when the wells are drilled and the GhG (biogas) extraction components are installed and placed in communication with the biodigester environment. It will be appreciated that these two time ranges in FIGS. 1 and 2 may be used to prepare the formulae for one or more compound additives to achieve the delayed bio-assimilation period as desired. Since the long time periods illustrated in FIGS. 1 and 2 between events D and E respectively, are about 23 years or more, it allows sufficient flexibility in formulation.

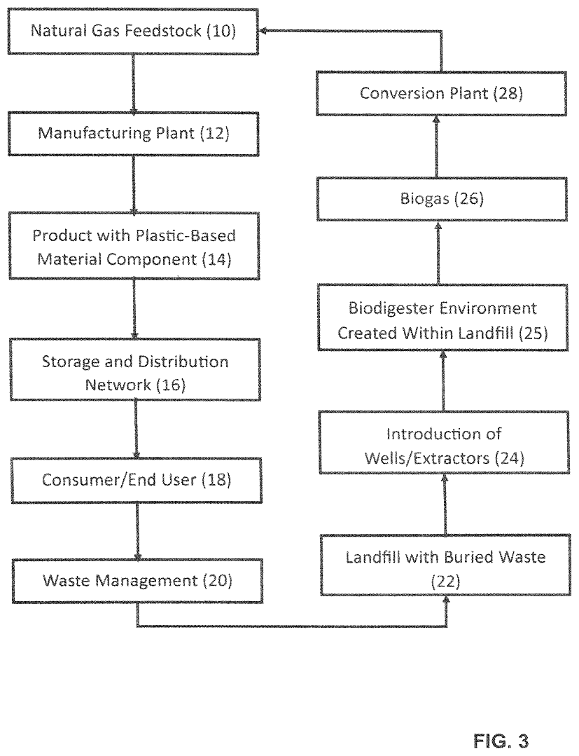

Referring now to FIG. 3 depicting a general overview of the components of a closed loop biogas recycling system, a petroleum based or natural gas feedstock 10 provides a material source for a product to be made that incorporates a plastic or polymer based material at a manufacturing plant 12. The product 14 is constructed with a plastic-based material component according to the processes and formulae described herein to have an inherent bio-assimilation dormancy period or “time bomb” characteristic. The product enters a distribution and storage network 16 until the product enters into actual use by a consumer/end user 18. Once the DEOL expires, the product 14 is disposed in a local waste collection where waste management 20 picks up and delivers to the landfill 22. The product (now trash) remains dormant in the landfill environment until the wells/extractors 24 have been drilled and placed in communication with the landfill environment exposing the buried trash to the elements and initiating a biodigester environment 25. With the wells drilled, bio-assimilation of the product (trash) begins and a volume of biogas 26 is produced within the landfill's newly created biodigester environment 22. The wells/extractors retrieve the volume of biogas during the biogas collection period. The collected biogas is transferred to a conversion plant 28 when the biogas may be converted into energy or back into the natural gas feedstock 10.

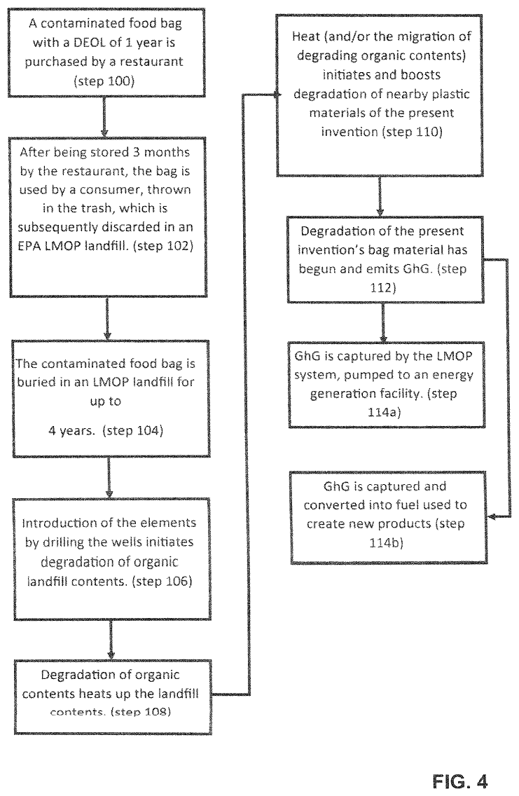

Referring now to the process shown in FIG. 4, the biogas production and extraction recycling stream begins with a contaminated food bag 14 (FIG. 3) constructed in accordance with the principles of the present invention. In this exemplary embodiment, it is understood that the contaminated bag 14 has an established DEOL of 1 year following a manufacturing date (event A in FIGS. 1 and 2). For example, a food bag may be purchased by a restaurant and subsequently used to store greasy French fries at a restaurant at step 100. The bag is initially stored for 3 months, then used by a consumer 18 (FIG. 3) eating in the restaurant, and ultimately discarded in the trash before being picked up by local trash collector who delivers the bag (now trash) to an EPA regulated LMOP landfill at step 102. The bag 14 is buried for up to 4 years along with other trash in step 104. During this 4 year timeframe the plastic-based material (typically, a long chain polymer) used to make the contaminated food bag remains dormant and does not bio-assimilate, nor biodegrade, nor degrade in any other manner, nor does it prematurely release greenhouse gas (GhG) to satisfy the requirements of the EPA. This refers to all of the plastic's composition regardless of organic, mineral, or chemical. Note it is the premature degradation of trash, primarily discarded paper, cellulose, and other organic and anaerobic plastic products during this four year period that prematurely releases GhG in the landfill. After 4 years the LFG wells are drilled at step 106, which releases built-up gases and initiates aerobic degradation of the organic contents within the buried regions of the landfill, contaminated food bag 14 along with the surrounding organic materials also begin fragmentation and bio-assimilation which heats up the landfill contents in step 108. Bio-assimilation of organic contents in landfills can produce heat up to 130 degrees at step 110, which boosts bio-assimilation of both organic materials and the bio-assimilation of the plastic-based material used to make contaminated bag 14 and the resulting heat and/or migration of the organic components in the landfill initiates and boosts bio-assimilation of nearby plastic materials also constructed in accordance with the principles of the present invention in step 110. In this example, the product incorporating at least one plastic-based material and constructed in accordance with the principles of the present invention includes additives, such as those identified herein, and are specifically formulated, to initiate bio-assimilation resulting in the production of biogas when warmer temperatures such as 130 degrees are reached in step 112. Somewhat cooler temperatures from 100 degrees and up, are also capable of inducing bio-assimilation of the plastic. However, this may take a longer time under the lower temperature. Likewise, the formulae may be adjusted to the cooler temperatures, as low as the high 80 degrees Fahrenheit. As previously described in FIGS. 1 and 2, the bio-assimilation process converts from aerobic to anaerobic two years after wells are drilled up to the remainder of EPA LMOP landfill's 30 year life. The biogas 26 (FIG. 3) is captured by the LMOP system and pumped to an energy generation facility in step 114a. Alternatively, or in addition thereto, the biogas may be captured and converted into fuel or as a feedstock to create new products 14 (FIG. 3) in step 114b.

This delayed initiation of the bio-assimilation of the plastic product 14 (FIG. 3) within 20 the landfill environment 22 is right at the crux of the LMOP landfill-fill safe technology: To formulate the use of salts, bioplastic and synthetic blends, organic biopolymers (such as chitin), molecular modification methods and additives, perhaps even enzymes, and so on, to initiate or cause bio-assimilation when the landfill heat rises to the warmer temperatures. The opposite may also be said by initiating bio-assimilation after the cooler temperatures of a covered landfill's contents not yet having wells drilled, have come to an end. The time-bomb effect after LFG wells are drilled may be activated by any number of time-, chemical-, mineral-, or organic-activated substances that cause the breakdown of the plastic-based material's long-chain molecular structure to allow sufficient fragmentation for bio-assimilation by common landfill microbes. Furthermore, in order for plastics that are predominantly bioplastics to safely degrade in LMOP 30 landfills, a form of “degradation preventative” or“chain extender” may be required during the first four years in the landfill, so thereafter the product can suitably be bio-assimilated after the wells are drilled. Various types of chemical and organic degradation preventative materials may be included in their structures, or it may be engineered into its bioplastic molecular structure before or during the compounding (resin forming) process.

Still referring to FIG. 4, during the heat-initiated timeframe (steps 108-110), fragmentation and bio-assimilation of the present invention bag 14 FIG. 3) is underway and LFG gases (primarily CO2 and methane biogases) are generated at step 112. These GhG gases are similar to those emitted by organic materials. It is also during this stage and thereafter that the formula of the present invention causes its plastic-based material to continue to sufficiently fragment and breakdown into tiny bio-available pieces. This process of safe bio-assimilation in EPA LMOP landfill is driven by the additive (or otherwise) formula with the appropriate “time bomb” effect-determined by the at least one year DEOL plus an additional 4 year biodigester dormancy time period as desired in the EPA Landfills.

As previously discussed in FIGS. 1 and 2, the contaminated food bag 14 total non-degradation time period (NDTP) of 5 years includes: A) Time spent before, and in, the manufacturing facility; B) the shipping and storage time frame to, and in, a distribution center; C) the time frame the product is shipped to, and stored in, the end user's restaurant; D) the time it takes before the product is used by the consumer and discarded in the restaurant trash, and; E) the time period the trash is delivered to the landfill facility, sent to the actual landfill, compacted (with other trash), buried and covered, which may be days or up to four years. Items A-D in the preceding list generally define the desired end of useful life (DEOL) time period while item E defines the up-to-four-year buried and covered burial dormancy time period, which together define the NDTP. Following the end of item E, in which the wells are drilled is referred to as the biodigester time period. Note that the DEOL time period and the buried and covered (burial dormancy) time period defining the NDTP may also be triggered by a temperature change instead of the combined time periods.

When biogas 26 (FIG. 3) is subsequently generated and captured in the LMOP landfill at step 114a, biogas 26 released by contaminated food bag 14 (FIG. 3) is sent through a network of pipes 24 (FIG. 3) to a generation plant 28 (FIG. 3). The methane gas is used as fuel to generate electricity, supply fuel to automobiles, or used in various alternative applications, the CO2 is typically released. Alternatively, the captured biogas 26 may be converted into a natural gas feedstock to produce new products in step 114b.

In the FIG. 4 example, the formula for the plastic-based material component of the product 14 (FIG. 3) incorporates an organic additive taking into account a one-year DEOL. However, with short term durables, for example household containers, the DEOL may be 5 years, thus the additional 4 years makes an NDTP of 9 years, requiring different formulae. Regardless of the DEOL, the formulae of the present invention are specifically formulated to have the desired NDTP providing it is no longer than about 20-25 years. An NDTP time allotment should include sufficient additional time to allow a product of the present invention to completely be bio-assimilated in the landfill—prior to the end of the 30-year landfill life.

There are four primary approaches that can accomplish successful bio-assimilation of plastic, most important, fossil fuel plastics in EPA regulated LMOP landfills. They are: 1) Use a catalyzer-mineral or organic—to break down the long molecular chains, so they may be bio-assimilated along with the organic contents in the landfill; 2) Use a catalyzer-mineral or organic-—to break down the long molecular chains, accompanied with an organic component, which gives the microorganisms food to initiate bio-assimilation; 3) Use an organic additive that serves to initiate bio-assimilation, and; 4) use an organic additive that may also serve as a catalyzer and subsequently as food for bio-assimilation. Where a food source is incorporated, the amount is between 0.25% to 3% of the total molecular weight or volume of the modified product composition. The use of a mineral catalyzer may be between 0.1% to 1% of the total molecular weight or volume of the modified product composition. These approaches are used to modify the composition of the product and/or plastic-based material component of the product 14 (FIG. 3).

One important consideration is to broadly disperse the additives throughout the fossil fuel plastic in order to have overall bio-assimilation, unlike the aggressive nature of oxos like Cadmium and Cobalt. Particle size under 2 microns is preferred and nano particles are superior. As cited by Dr. Joseph Greene, and by Bradfom LaPray in U.S. Patent Application No. US 2020/0339784 (the ′784 application) nano particle dispersion is preferred for bio-assimilation of plastic. There is another benefit to the use of smaller particle sizes, which is it lowers the amount of catalyzer or food, or both, required to initiate and complete bio-assimilation of the plastic. Thus, it lowers the cost. But this lower amount/cost phenomena only applies to plastics being disposed in LMOP landfills. As cited in the ′784 application, and as commonly pursued in the plastics trade with obsessive degradation (or biodegradation as the case may be) from Oxos or otherwise, generally higher quantities are used in order to make “biodegradation” claims (referring to the requirements of FTC Environmental Marketing Claims—Part 260—aka Green Marketing claims). Biodegradation is exactly what the present invention does NOT want.

On the other hand, it is preferable to convert the plastic into biogas which may further be processed into either energy or RNG. As opposed to aggressive biodegradation in a short time period—the environment in an EPA LMOP landfill has up to 30 years to be bio-assimilated. That is a substantial time-period, one completely ignored in the plastics trade-again which pursues rapid degradation in weeks or months. This long time frame significantly reduces the amount of catalyzer required, if any at all, and the amount of organic food that helps initiate bio-assimilation.

The significant reduction in the amount of catalyzer does something else of great importance for the bio-assimilation deferred processes described herein. The reduction in the amount of catalyzer can overcome short-term degradation issues during the first 5 years, and the premature release of GhG in the LMOP landfills. Very little, if any catalyzer is needed. This is especially true during those first four years as the contents in the landfill are compacted in cooler and cold environments, further assisting in preventing (contributing) to premature degradation release of GhG. As a comparison, Oxos used at its typical 3% rate create uneven rapid degradation of the plastic, unless higher rates are used; whereas the plastic made in accordance with the principles of the present invention can be at a rate of 0.1% to 1% of the total molecular weight or volume of the modified product composition. Obviously, there is a substantial cost savings for the plastic manufacturer producing products made in accordance with the principles of the present invention, and with far superior results—by completing a true closed loop recovery at the EPA LMOP landfill. Furthermore, it does it all by simply using the existing waste management infrastructure.

Furthermore, it is preferred to have food contaminated plastics that have not been cleaned, and paper/poly laminated films, whereas a paper laminate provides food for the future bio-assimilation of the present invention in an EPA landfill. Thus, products of this sort may require lower portions of additives to kickstart and conclude bio-assimilation. The overall NDTP and subsequent bio-assimilation period vitally depend on a product's initial use, the product's performance during the early burial stages, and then contributing to anaerobic bio-assimilation after the two-year aerobic digestion period. While there are 100+ market segments for plastics, each segment and application requires an initial DEOL use and preferably a level of food contamination.

Note this same DEOL principle may be applied to films that have sun exposure as, for example, mulch films and other used to protect against frost, such as winery vines. Solar degradation initiating the break-up of a plastic's molecules gets a head start on others, whereas a lesser amount of catalyzer or additive is required.

Generally speaking, additives with heavy starch content such as sorghum, potatoes, tapioca, agave, and so on are preferred, and can more easily be calculated for suitable formulae percentages depending on the plastic and application. Corn starch processing requires additional processing steps and can affect food prices, thus is not as appealing, however, it too can be used.

Simply put, the plastic constructed in accordance with the principles of the present invention may use various catalyzer and thermal substitutes to produce the breaking down the long molecular chains, making them more readily bio-assimilated by common landfill microbes. Likewise, certain types of organic additives can also be formulated whereas no catalyzer is required. This is particularly true considering the long 30 year life of a biogas producing LMOP landfill. More common is a combination of: 1) a catalyzer, and 2) an organic component—used as food by the microorganisms.

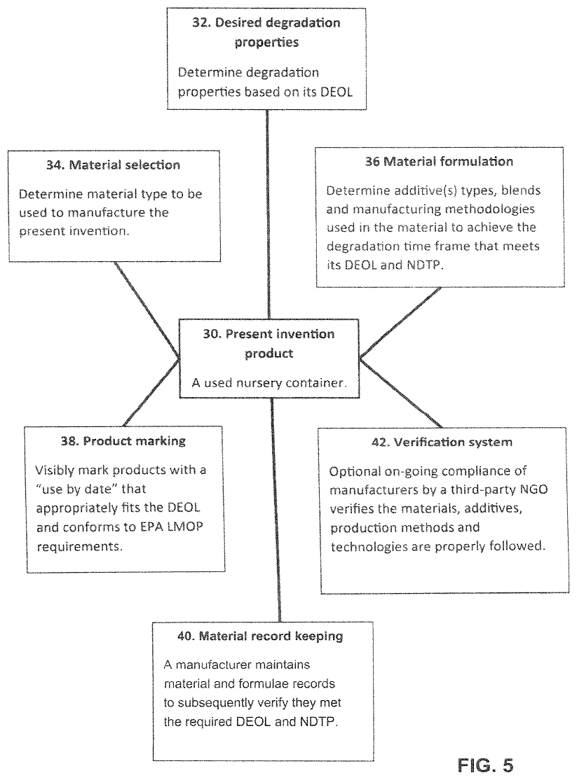

Turning now to the block diagram of FIG. 5, a set of requirements for using a product constructed in accordance with the principles of the present invention including formulating, testing, marking, and verification processes is depicted. An exemplary nursery container 30 constructed in accordance with the principles of the present invention has a 2 year DEOL and is one container in a much larger production run of thousands of containers. The production run typically uses existing plastics manufacturing processes and machinery. Prior to manufacturing the production run, the desired bio-assimilation properties 32 of nursery container 30 have been determined based on a 6 year NDTP (2 year DEOL and 4 year LMOP non-degradation period). If the DEOL of nursery container 30 is used to grow plants in a longer timeframe at a farm, then perhaps a 7 year NDTP is preferable. It is generally safer to calculate a longer NDTP than a shorter one. For nursery container 30, a compatible plastic material 34 using the 6 year NDTP formula is selected. In this case, the material formulation 36 includes a synthetic polypropylene material with a dark green colorant and a mineral and bio additive that gives the 6 year time-bomb effect. This material formulation 36 may also consider if the container is used to grow plants outdoors or indoors. With long periods growing outdoor plants, it may include a chain extender or UV inhibitor.

During the manufacture of nursery container 30, either it or its outside wrapper has a printed product marking 38 in the form of a legal notice declaring: A) Do not recycle, and; B) Safe to dispose in EPA LMOP landfills that convert decomposing trash into energy. Or anything similar that is easy for users to understand. It may also include a “use by date,” similar to those used with foods, for example, “sell by 7-14-22”, which “use by date” is typically the end of the DEOL. These types of notices may also include ASTM numbers, manufacturer's name, relevant state laws, and country of origin. A product's manufacturer should also keep all manufacturing records 40, should they be required to verify the formula used was correctly applied. A government agency or an optional 3^{rd} party NGO 42 may also be used to certify that a manufacturer is in compliance with all formulae, early non-degradation qualities and later landfill bio-assimilation properties within the allotted time frame of the established life of an EPA LMOP landfill. As previously stated, an EPA LMOP landfill has a thirty year maximum life after wells are drilled and gas extraction begins, whereas after twenty-five years profitability wanes. It is an object of the present invention to have its resultant products to be fully bio-assimilated by about 25 years after well-drilling.

B. Description of Formulae Determinations, Components, and their Related Processes

It is clear that the amount of catalyzers and organic compounds incorporated during the manufacturing process to modify the plastic-based material components used to create products constructed in accordance with the principles of the present invention rely on several time-sensitive factors. Likewise, the properties in the compounds rely on other factors, for example, particle size, molecular structure, as well as the type of organic and inorganic substances used to make them. With catalyzers and organic food sources, particle sizes less than 2 microns are preferred, with superior results in nano-particle ranges. The smaller the particle size, the more even the dispersion, the easier it is for microbes to “nibble” the plastic in their natural quest to bio-assimilate the material. The more even the dispersion, the more complete the molecular breakdown and subsequent bio-assimilation. While particle sizes larger than 2 microns can work with the present invention, it may result in uneven bio-assimilation. However, with the long-term target of 25 years in an active LMOP landfill, it may not matter anyway. Migration also tends to play a part in allowing catalyzer properties to spread throughout a plastic. Iron, in particular an iron stearate, has nano-particle properties, which are advantageous for several reasons. It is commonly viewed as being a non-toxic mineral and found everywhere in soil. Likewise, it is abundant in North America for its large market. While cobalt can also be made to work well, it is not abundant in the US, and controlled by foreign countries outside the U.S. Being banned in some states makes it less desirable, regardless of whether the bans are scientifically justified or not. Obvious cost is a factor as well.

One very promising catalyzer that may be used is calcium oxide, CaO. It can inexpensively be manufactured through pyrolysis of calcium carbonate, CaCO3, at 700 degrees F. CaCO3 is abundant worldwide, commonly mined from limestone, may be sourced from seashells, such as clams and oysters, very inexpensive, and favorably viewed by the public.

Organic substances can be made to have catalytic properties, but most organic catalyzers in the market today tend to have at least a small amount of mineral content to initiative oxidation and break down of the molecular chains. With the present invention it makes no difference if a supposed organic catalyzer compound is wholly or partially organic as long as it provides safe bio-assimilation as desired.

Organic substances used as food to assist in bio-assimilation are also affected by particle size. Like a catalyzer, the smaller the particle size, the superior the particle dispersion and subsequent bio-assimilation. It is important to note that no catalyzer would be required should the organic particle size be sufficiently small and blended into the plastic in generally larger quantities, for example 10% or more. Once again, with the long 25 year bio-assimilation time period in an EPA LMOP landfill, even smaller percentages may be sufficient, as small at 0.25% if it is a more bioactive plastic such as PETG.

The actual amount or catalyzer or organic food component used in products constructed in accordance with the principles of the present invention depends on the plastic type, product, and so on, and may be as low as 1/10^{th} of one percent (0.25%), or as high as 1%. The percentage is further complicated by the carrier—the raw plastic material-used in the compound and its percentage. Compound carriers are typically in the 25% to 60% range, with most around 50%. Therefore, a compound with 25% carrier contains about 70% catalytic substance and 5% wax and other surfactant substances. A carrier as high as 60% would contain about 35% catalytic substance and 5% wax, etc. The ideal in the trade would be to have a nano-particle catalyzer and/or organic food substance in the same compound with as little carrier as possible.

### C. Variations

Herein, a somewhat complicated, however specific, and unique approach to create plastic products that can be effectively manufactured and used, and likewise meet the US EPA LMOP requirements when disposed in landfills has been disclosed. The non-degradation and bio-assimilation properties required in the plastics and products constructed in accordance with the principles disclosed herein may be accomplished in several ways, many that are not illustrated herein, and; many that will be discovered in the future. Almost invariably, the primary material component in the plastics and products constructed in accordance with the principles disclosed herein is a common synthetic such as polyethylene, polypropylene, ABS, PET, and so on, or at times with bio-based plastics. The synthetic and/or bio-based materials may be made with or without recycled or virgin content or in combination thereof, manufactured on various types of machinery with various types of processes, and with essentially the same results that meet EPA LMOP landfill requirements.

The principles cited herein may also apply to some energy from waste (EFW) applications and essentially all agriculture biogas plants. The primary difference is that a speedier bio-assimilation period is preferred. This is accomplished by adjusting the catalyzer and organic components as desired. In such an application, it may be preferred to use no organic component as it literally is being treated in an “organic bath” of ag waste at the facility.

Note too that when employed in other countries and continents (outside the United States), for example Indonesia and Asia, they may have shorter 2 or 3 year non-degradation timeframes in their LFG landfills. Most trash taken to a US landfill takes only a few days to arrive, thus the short timeframe has little or no impact on the calculations used herein, but in some foreign countries, it may take a long time to reach a landfill, and therefore should be taken into consideration. Plus, landfills worldwide contain varying amounts of pure garbage, which is not contained in U.S. landfills. Formulae of the present invention used to: 1) govern an initial non-degradation timeframe (generally 4 years in the U.S.); 2) create a time-bomb effect to safely initiate bio-assimilation after the initial non-degradation timeframe when wells are drilled and aerobic bio-assimilation begins; 3) continuously bio-assimilate during the anaerobic stage, and; 4) leave behind no toxic waste or microplastics is the desired outcome. This doable objective may be effectively accomplished by using the various blended or homogenous resins, bio or mineral salts, processing agents and other technologies as cited herein. Optional color additives and processing agents may also be incorporated. Large resin manufacturers may incorporate additives and/or molecular modifications in its plastic reactor process and subsequent processes that contain catalytic effects later on, in the landfill environment.

One optional process is to use: A) a controllable non-toxic mineral additive, such as cobalt, along with; B) a processing agent such as CaCO3 (either mined or extracted from seawater such as oyster and clam shells, which are crushed into a powder) and then; C) mix in organic component, such as any number of organic materials that have been compounded into a bioplastic. Other additives that may be substituted for the organic component are chitin and chitosan, also known as biopolymers. Likewise, a preferred process for large volume production may be created without the use of any additive, but solely molecular modification pre-embedded in the plastic resins. The use of chitin may be sourced from marine shells, which is usually about 95% CaCO3 and 5% chitin. Ground up and used in the present invention formulae in desired amounts, it may be used as both a CaCO3 processing component and a biopolymer bio-assimilation enhancing component. Chitin and other mineral and bio salts may also be included in color additives or other processing agents to achieve the desired outcome.

The manufacturers of these types of additives, processing agents, and molecular modification technologies singularly and in combination maintain close trade secrets, and so do users and manufacturers. This includes proprietary raw material suppliers, mineral grinding techniques, bio raw materials and their compounding processes, and so on. They may include specific percentages of use in any given formula depending on the DEOL and the NDTP. There is no one-size-fits all, like the approach employed by many oxo salt suppliers using cadmium or other minerals, or degradant accelerant suppliers using bio-based and enzyme additives. In many cases the cost of the additives cited herein would raise the cost of the final products' sell price by as little as 2%-4%.

Other variations for modifying the product composition of a product constructed in accordance with the principles of the present invention may be to include a larger amount of CaCO3 to help avoid present day recycling and cleaning operations in recycling plants. For example, polyethylene floats with less than 12% CaCO3, and sinks when in higher percentages. During the initial washing process of polyethylene films in the recycling process, a non-recyclable polyethylene product of the present invention could be made to sink and be discarded. Again, it is the preference to mark all such products with a notice reading, “Do not recycle,” however if it happens to slip through and get deposited in a recycling bin, it may be easily removed later.

It will be appreciated that applicant has invented a new and useful methodology to determine: 1) a product's useful end of life; 2) safe bio-assimilation in an EPA regulated landfills without the premature release of Greenhouse gas (GhG); 3) capturing both methane and GhG gases and converting it into energy or a renewable product feedstock, and; 4) create a closed loop recovery methodology (i.e., recycling). It also includes methods for manufacturers and users to create and employ the products constructed in accordance with the principles of the present invention as well as a method to verify proper use.

The spirit of the present invention provides a breadth of scope that includes all methods of making, formulating, and use. Any variation on the theme and methodology of accomplishing the same outcome that are not described herein would be considered under the scope of the present invention.

Certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Although this invention has been disclosed in the context of certain preferred embodiments and examples, it will be understood by the few who are skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. Nevertheless, the principal teachings, in particular in FIGS. 1 and 2, are ideal to create test 5 protocols used to verify and/or create a new ASTM standard using multiple testing points for charting bio-assimilation of a plastic of the present invention, using: 1) ASTM D5296 and D882 to establish DEOL properties; 2) ASTM D5338 to verify aerobic bio-assimilation activity, and; 3) D5511 or D5526 to test anaerobic bio-assimilation, and production of biogas.

It is also contemplated that various combinations or sub-combinations of the specific features and aspects of the embodiments may be made and still fall within the scope of the invention. Accordingly, it should be understood that various features and aspects of the disclosed embodiments may be combined with or substituted for one another to form varying modes of the disclosed invention. Thus, it is intended that the scope of the present invention herein disclosed should not be limited by the particular disclosed embodiments described above.

What is claimed is:

1. A method of producing one or more biogases within a biodigester environment, the method comprising:
determining a non-degradation time period for a selected product to be manufactured and used with at least one plastic-based material component; and
modifying a product composition of the selected product during a manufacturing process to initiate bio-assimilation of the plastic-based material component of the selected product and subsequent production of at least one biogas after the product is disposed within the biodigester environment and the non-degradation time period expires.

2. The method of claim 1 wherein:
the biodigester environment is created within an LMOP landfill.

3. The method of claim 1 wherein:
the bio-assimilation of the plastic product within the biodigester environment results from both an aerobic process and an anaerobic process.

4. The method of claim 1 wherein:
the manufacturing process of the product is selected from a group consisting of one of the following machines: an extruder for blown film, a blow molder, a thermoformer, a cast film machine, a compression compounding machine, and an injection molder.

5. The method of claim 1 wherein:
a product composition modifier for modifying the product composition of the selected product is selected from a group consisting of at least one: salt, organic catalyzer, mineral catalyzer, bioplastic and synthetic blend, organic biopolymer, molecular modification, and an enzyme.

6. The method of claim 1 wherein:
the selected product is categorized as non-recyclable.

7. The method of claim 1 wherein:
the selected product is contaminated with an amount of organic material prior to placement within the biodigester environment.

8. The method of claim 1 wherein:
the selected product is constructed of dissimilar materials.

9. The method of claim 1 wherein:
the selected product is selected from a desired end of useful life duration group consisting of: a single use disposable product, a short term durable product, and a long term durable product.

10. The method of claim 1 wherein:
the end of the non-degradation time period is determined by a shift from a predetermined lower temperature to a predetermined higher temperature within the biodigester environment to trigger the production of the at least one biogas within the biodigester environment.

11. The method of claim 1 wherein:
the selected product is composed of a bioplastic including a chain extender to meet the non-degradation time period.

12. The method of claim 1 wherein:
the non-degradation time period is determined by calculating a desired end of useful life time period for the selected product and a burial dormancy time period in which the product is disposed within a burial environment prior to the transition to the biodigester environment.

13. The method of claim 12 wherein:
the desired end of useful life time period of a selected product is defined by a time period starting with a manufacturing date, continuing with use of the product in an ambient environment outside the burial environment, and ending with the product entering into the biodigester environment; and
the burial dormancy time period is defined by the initial disposition of the product within the burial environment lasting until one or more biogas extractors is placed in communication with the burial environment thereby initiating a biodigester environment.

14. The method of claim 12 wherein:
the dormancy time period includes an initial stage in which the selected product is sequestered within the burial environment having a first temperature and inhibiting the premature release of the at least one biogas and ends during a first transition stage in which the burial environment heats up sufficiently to instigate bio-assimilation of the plastic-based material of the selected product.

15. The method of claim 14 wherein:
a shift in temperature above the first temperature is triggered by placing one or more biogas extractors in communication with the burial environment to initiate transition to the biodigester environment.

16. The method of claim 12 further comprising:
transitioning to a higher temperature environment than the temperature of the burial environment at the end of the burial dormancy time period resulting in bio-assimilation of the plastic-based material arising from an aerobic or anaerobic process converting the plastic-based material into a volume of at least one biogas;
capturing at least a portion of the volume of biogas; and
converting the captured volume of biogas into an energy source for storage and subsequent use.

17. The method of claim 12 further comprising:
determining a desired bio-assimilation time period in which the biodigester environment is undergoing an anaerobic process resulting in the production of a volume of biogas, the bio-assimilation time period occurring after the desired burial dormancy time period expires and lasting through an end of a useful biogas extraction event from the biodigester environment by one or more biogas extractors placed in communication with the biodigester environment.

18. The method of claim 12 wherein:
the desired end of useful life time period is one year; and
the total non-degradation time period is five years.

19. The method of claim 12 further comprising:
determining a biogas collection time period following expiration of the burial dormancy time period running from the initial placement of one or more biogas extractors placed in communication with the burial environment until collection of at least one biogas resulting from the bio-assimilation of the plastic-based materials in the selected products is substantially completed; and
collecting a volume of biogas generated by the bio-assimilation of the plastic-based material component of the selected product during the biogas collection time period.

20. The method of claim 12 wherein:
the product composition is modified by introducing an additive during the manufacturing process of the selected product, the additive having a negligible impact on the desired end of useful life time period with the product disposed within an ambient environment while also delaying production of at least one biogas resulting from bio-assimilation of the plastic-based material component of the selected product until the product reaches the end of the overall useful life and disposition in the burial environment and enters into the biodigester environment.

21. The method of claim 20 wherein:
the additive is selected from a group consisting of: sorghum, potato, tapioca, agave, corn starch, avocado, and beets.

22. The method of claim 20 wherein:
the additive is organic.

23. The method of claim 20 wherein:
the additive is inorganic.

24. The method of claim 20 wherein:
the additive is a biopolymer selected from a group consisting of: chitin, chitosan, a commercially manufactured biopolymer sold under the tradename Ecoplas™, and a commercially manufactured biopolymer sold under the tradename Ingeo™.

25. The method of claim 20 wherein:
the additive functions as both a catalyzer and a digestible substance to be consumed by one or more microorganisms disposed within the biodigester environment.

26. The method of claim 20 wherein:
the additive includes at least one organic or inorganic substance that serves as a food source for common microbes found in a landfill or a biodigester.

27. The method of claim 26 wherein:
the food source is between 0.25% to 3% of the total molecular weight or volume of the modified product composition.

28. The method of claim 1 further comprising:
introducing a catalyzer into the product composition of the selected product during the manufacturing process, the catalyzer being selected to break down the long molecular chains of the plastic-based material component of the product composition within the biodigester environment.

29. The method of claim 28 wherein:
the catalyzer is accompanied by an organic component providing a digestible material to be consumed by one or more microorganisms disposed within the biodigester environment.

30. The method of claim 28 wherein:
the catalyzer causes the long molecular chains of the plastic-based material to breakdown when exposed to heat.

31. The method of claim 28 wherein:
the catalyzer is selected from a group consisting of: iron, iron stearate, cobalt, cadmium, calcium, magnesium, calcium oxide, and calcium carbonate.

32. The method of claim 28 wherein:
the catalyzer is between 0.1% to 1% of the total molecular weight or volume of the modified product composition.

33. A polymer for use in the manufacturing of a product constructed to enter a biodigester environment following the expiration of a desired end of a useful life time period and a subsequent burial dormancy period, the polymer comprising:
at least one plastic-based material component; and
an additive introduced during the manufacturing process of the product, the additive inhibiting degradation of the at least one plastic-based material component during the desired end of useful life time period and the burial dormancy time period, the additive further enhancing bio-assimilation of the plastic-based material component once the biodigester heats up to create the biodigester environment.

34. The polymer of claim 33 wherein:
the additive includes at least one organic component.

35. The polymer of claim 33 wherein:
the additive includes at least one inorganic component.

36. The polymer in claim 33 wherein:
the additive is composed of at least one organic catalyzer.

37. The polymer of claim 33 wherein:
the additive is composed of at least one inorganic catalyzer.

38. The polymer in claim 33 wherein:
the additive includes at least one food source digestible by microbes present in the biodigester.

39. A process for modifying a polymer during the manufacturing process of a product to initiate bio-assimilation of the product within a biodigester environment, the process comprising:
selecting a product to be manufactured;
selecting a polymer to be used in the manufacturing process of the product;
determining a first product lifecycle stage defined by use of the product in an ambient environment external to the biodigester environment;
determining a second product lifecycle stage defined by the initial entry of the product into a burial environment and;
determining a third product lifecycle stage defined by initiation of bio-assimilation of the product by either an aerobic or anaerobic process within the biodigester environment;
determining a fourth product lifecycle stage in the biodigester environment during which anaerobic processes convert the polymer into a volume of one or more biogases; and
modifying the polymer during the manufacturing process of the product to account for all four lifecycle stages.

40. A closed loop biogas collection system comprising:
a biodigester defining a biogas generating environment;
a plurality of products disposed within the biogas generating environment, each product having an expired desired end of useful life time period during which the product was previously used in an ambient environment external to the biogas generating environment, the products being constructed of at least one plastic-based material and further constructed with a built-in bio-assimilation resistance time period of the product following initial disposition within the biogas generating environment and entering a bio-assimilation period following the expiration of the bio-assimilation resistance period resulting in complete bio-assimilation of the product along with the production of a volume of biogas; and
a biogas extraction component placed in communication with the biogas generating environment and constructed to extract a volume of biogas resulting from the bio-assimilation of the plastic-based material lasting until a predetermined biogas volume capture level is obtained.

41. A polymer based composition for delaying bio-assimilation and production of biogas within a biodigester, the polymer based composition comprising:
a polymer with a long molecular chain structure resistant to degradation and bio-assimilation; and
an additive modifying the molecular chain structure of the polymer to produce a modified polymer during the manufacturing process, the additive being defined by a quantity and a type that has a negligible effect on the degradation of the modified polymer during a desired end of useful life time period in an ambient environment, allows the modified polymer to remain dormant until such time as the biodigester environment begins, and also assists in the bio-assimilation of the modified polymer and the subsequent generation ofa volume of biogas resulting from the breakdown of the modified polymer after one or more biogas extractors are placed in communication with the biodigester.

42. A process to modify a long chain molecular structure of a plastic resin in a reactor comprising:
introducing a plastic resin into a reactor; and
adding a heat sensitive molecular component to be blended with the plastic resin in the reactor, the heat sensitive molecular component being responsive to a high heat environment of at least one hundred degrees Fahrenheit to initiate the breakdown of the long chain molecular structure resulting in a plurality of shorter molecular chains that may be bio-assimilated by common microbes present in a biodigester environment when the plastic resin is placed therein.

43. The process in claim 42 wherein:
the plastic resin is compounded with one or more organic or inorganic substances soon after creation in the reactor.

44. A method to test and validate bio-assimilation of a modified polymer in the following order, comprising:
testing a first lifecycle stage of use of the modified polymer in a simulated ambient environment to verify a low threshold level of degradation;
testing a second lifecycle stage in which the modified polymer is buried in a simulated burial environment to verify a low threshold release of a volume of biogas;
testing a third lifecycle stage in which the modified polymer is placed in a simulated biodigester environment to verify bio-assimilation of the modified polymer, which is initiated by an aerobic process, and;
testing a fourth lifecycle stage in which the modified polymer remains in a simulated biodigester environment to verify bio-assimilation of the modified polymer is converted into a volume of one or more biogases in an anaerobic process.

45. A method to prevent premature bio-assimilation of a modified polymer until a biogas capturing device is put in place to capture one or more biogases, comprising:
disposing the modified polymer in a sequestered cool environment inhibiting the premature bio-assimilation of the modified polymer to prevent the release of a volume of biogas into the atmosphere:
transitioning the cool environment to a higher temperature to initiate bio-assimilation of the modified polymer due to the introduction of one or more biogas capturing devices; and
transitioning from an aerobic process to an anaerobic process within the higher temperature environment resulting in the production of a volume of biogas as the modified polymer is broken down.

46. The method of claim 45 further comprising:
entering a biogas capturing stage defined by collection of a volume of one or more biogases by the one or more biogas capturing devices;
converting the captured volume of one or more biogases into an energy source; and
storing the energy source for subsequent use.

47. The method of claim 46 further comprising:
storing the captured volume of one or more biogases as renewable natural gas.

48. The method in claim 47 wherein:
manufacturing a plastic resin in a plastic reactor using at least some of the renewable natural gas in a plastic reactor.
